(19) United States
(12) Patent Application Publication
(10) Pub. No.: US 2016/0128999 A1
(43) Pub. Date: May 12, 2016

(54) MASITINIB FOR TREATING HEPATIC CANCER

(71) Applicant: AB SCIENCE, Paris (FR)

(72) Inventors: Alain MOUSSY, Dauphine (FR); Jean-Pierre KINET, Lexington, MA (US)

(21) Appl. No.: 14/939,712

(22) Filed: Nov. 12, 2015

(30) Foreign Application Priority Data
Nov. 12, 2014 (EP) 14192924

(51) Int. Cl. A61K 31/496; A61K 31/704; A61K 31/4745; A61K 31/357; A61K 31/7068; A61K 31/475

(52) U.S. Cl. CPC A61K 31/496; A61K 31/7068; A61K 31/475; A61K 31/4745; A61K 31/357; A61K 31/704

(57) ABSTRACT

A method for treating hepatic cancer in a subject in need thereof, which includes the administration to the subject a therapeutically effective amount of a tyrosine kinase inhibitor, in combination with a therapeutically effective amount of a chemotherapeutic agent.

### FIELD OF INVENTION

The present invention relates to the treatment of hepatic cancer. More specifically, the present invention relates to the treatment of hepatocellular carcinoma, using masitinib.

### BACKGROUND OF INVENTION

Hepatic cancer (or liver cancer) is a cancer that originates in the liver, and may be referred to as primary hepatic cancer, with opposition to liver metastases that originate from organs elsewhere in the body and migrate to the liver (liver metastases are also referred to as secondary hepatic cancer).

Primary hepatic cancers are formed from either the liver itself (and are named hepatocellular carcinoma), or from structures within the liver (including blood vessels or bile duct (leading to cholangiocarcinoma)).

Hepatocellular carcinoma (HCC) is the fifth most common cancer and the third most common cause of cancer-related death worldwide with 600 000 patients dying of this disease every year. HCC occurs in the setting of underlying liver diseases, including hepatitis B and C, non-alcoholic fatty acid liver disease, and alcohol-induced cirrhosis. HCC is potentially curable by surgical resection but surgery is the treatment of choice for only the small fraction of patients with localized and resectable tumor or cancer.

Until recently, there were no systemic options that clearly improved survival for patients with advanced unresectable HCC. Clinical studies evaluating the use of chemotherapy (doxorubicin or the combination of gemcitabine and oxaliplatin (GEMOX)) have reported unsatisfactory low response rate and no benefit in terms of overall survival. Therefore new therapeutic options are urgently needed.

Hepatocellular carcinoma is a highly vascularized tumor, which makes vascular targeting approaches particularly appealing for the treatment of HCC. Tumour angiogenesis is mediated mainly by vascular endothelial growth factor (VEGF) produced by tumoural cells that activates VEGF receptors (VEGFR) on surrounding endothelial cells concomitant to the activation of receptors of platelet-derived growth factor (PDGF) on pericytes. Antiangiogenic drugs such as sorafenib and bevacizumab have already shown significant clinical activity in HCC, and sorafenib is now the FDA-authorized agent for patients with advanced HCC. However, the benefit of these new targeted therapies in terms of overall survival remains relatively modest and safety concerns have been raised. Indeed, most of these agents have been recently associated with toxicity to the heart and bowel perforation; especially hypertension and thromboembolic phenomenon were observed in patients treated with bevacizumab. Therefore, there are still great needs for new therapeutic strategies using safer and more efficient targeted agents to improve cancer treatment and to circumvent resistance to chemotherapeutic agents.

Masitinib is a novel tyrosine kinase inhibitor of the 2-aminoarylthiazoles derivatives family, that mainly targets c-Kit and the angiogenic PDGF receptors but was also found to target the non-receptor tyrosine kinases Lyn and Fyn and to a lower extent FGFR3 (Dubreuil et al. 2009).

The Applicant herein surprisingly demonstrates that Masitinib potentiates the cytotoxic effect of chemotherapies in HCC, including gemcitabine, doxorubicin, irinotecan, etoposide and vincristine. The present invention thus relates to the synergistic combination of masitinib and at least one chemotherapeutic agent for treating hepatic cancer.

### DEFINITIONS

In the present invention, the following terms have the following meanings:

The term “subject” refers to a mammal, preferably a human. In one embodiment, a subject may be a “patient”, i.e. a warm-blooded animal, more preferably a human, who/which is awaiting the receipt of, or is receiving medical care or was/is/will be the object of a medical procedure, or is monitored for the development of a hepatic cancer. In one embodiment, the subject is an adult (for example a subject above the age of 18). In another embodiment, the subject is a child (for example a subject below the age of 18). In one embodiment, the subject is a male. In another embodiment, the subject is a female.

The terms “treating” or “treatment” refers to both therapeutic treatment and prophylactic or preventative measures; wherein the object is to prevent or slow down (lessen) hepatic cancer. Those in need of treatment include those already with hepatic cancer as well as those prone to have hepatic cancer or those in whom hepatic cancer is to be prevented. A subject is successfully “treated” for hepatic cancer if, after receiving a therapeutic amount of a tyrosine kinase inhibitor according to the methods of the present invention, the patient shows observable and/or measurable reduction in or absence of one or more of the following: reduction in the number of pathogenic cells; reduction in the percent of total cells that are pathogenic; and/or relief to some extent, of one or more of the symptoms associated with hepatic cancer; reduced morbidity and mortality, and improvement in quality of life issues. The above parameters for assessing successful treatment and improvement in the disease are readily measurable by routine procedures familiar to a physician.

The term “therapeutically effective amount” means the level or amount of agent that is aimed at, without causing significant negative or adverse side effects to the target, (1) delaying or preventing the onset of hepatic cancer; (2) slowing down or stopping the progression, aggravation, or deterioration of one or more symptoms of hepatic cancer; (3) bringing about ameliorations of the symptoms of hepatic cancer; (4) reducing the severity or incidence of hepatic cancer; or (5) curing hepatic cancer. A therapeutically effective amount may be administered prior to the onset of hepatic cancer, for a prophylactic or preventive action. Alternatively or additionally, the therapeutically effective amount may be administered after initiation of hepatic cancer, for a therapeutic action or maintenance of a therapeutic action.

The term “pharmaceutically acceptable carrier or excipient” refers to an excipient or carrier that does not produce an adverse, allergic or other untoward reaction when administered to an animal, preferably a human. It includes any and all solvents, dispersion media, coatings, antibacterial and antifungal agents, isotonic and absorption delaying agents and the like. For human administration, injected preparations should meet sterility, pyrogenicity, general safety and purity standards as required by regulatory offices, such as, for example, FDA Office or EMA.

The term “about” preceding a figure means plus or minus 10% of the value of said figure.

As used herein, the term an “aryl group” means a monocyclic or polycyclic-aromatic radical comprising carbon and hydrogen atoms. Examples of suitable aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. An aryl group can be unsubstituted or substituted with one or more substituents. In one embodiment, the aryl group is a monocyclic ring, wherein the ring comprises 6 carbon atoms, referred to herein as “(C6)aryl”.

As used herein, the term “alkyl group” means a saturated straight chain or branched non-cyclic hydrocarbon having from 1 to 10 carbon atoms. Representative saturated straight chain alkyls include methyl, ethyl, n-propyl, n-butyl, n-pentyl, n-hexyl, n-heptyl, n-octyl, n-nonyl and n-decyl; while saturated branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like. Alkyl groups included in compounds of this invention may be optionally substituted with one or more substituents.

As used herein, the term “alkoxy” refers to an alkyl group which is attached to another moiety by an oxygen atom. Examples of alkoxy groups include methoxy, isopropoxy, ethoxy, tert-butoxy, and the like. Alkoxy groups may be optionally substituted with one or more substituents.

As used herein, the term “heteroaryl” or like terms means a monocyclic or polycyclic heteroaromatic ring comprising carbon atom ring members and one or more heteroatom ring members (such as, for example, oxygen, sulfur or nitrogen). Typically, a heteroaryl group has from 1 to about 5 heteroatom ring members and from 1 to about 14 carbon atom ring members. Representative heteroaryl groups include pyridyl, 1-oxo-pyridyl, furanyl, benzo[1,3]dioxolyl, benzo[1,4]dioxinyl, thienyl, pyrrolyl, oxazolyl, imidazolyl, thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, imidazo[1,2-a]pyridyl, and benzo(b)thienyl. A heteroatom may be substituted with a protecting group known to those of ordinary skill in the art, for example, the hydrogen on a nitrogen may be substituted with a tert-butoxycarbonyl group. Heteroaryl groups may be optionally substituted with one or more substituents. In addition, nitrogen or sulfur heteroatom ring members may be oxidized. In one embodiment, the heteroaromatic ring is selected from 5-8 membered monocyclic heteroaryl rings. The point of attachment of a heteroaromatic or heteroaryl ring to another group may be at either a carbon atom or a heteroatom of the heteroaromatic or heteroaryl rings.

The term “heterocycle” as used herein, refers collectively to heterocycloalkyl groups and heteroaryl groups.

As used herein, the term “heterocycloalkyl” means a monocyclic or polycyclic group having at least one heteroatom selected from O, N or S, and which has 2-11 carbon atoms, which may be saturated or unsaturated, but is not aromatic. Examples of heterocycloalkyl groups include (but are not limited to): piperidinyl, piperazinyl, 2-oxopiperazinyl, 2-oxopiperidinyl, 2-oxopyrrolidinyl, 4-piperidonyl, pyrrolidinyl, hydantoinyl, valerolactamyl, oxiranyl, oxetanyl, tetrahydropyranyl, tetrahydrothiopyranyl, tetrahydropyrindinyl, tetrahydropyrimidinyl, tetrahydrothiopyranyl sulfone, tetrahydrothiopyranyl sulfoxide, morpholinyl, thiomorpholinyl, thiomorpholinyl sulfoxide, thiomorpholinyl sulfone, 1,3-dioxolane, tetrahydrofuranyl, dihydrofuranyl-2-one, tetrahydrothienyl, and tetrahydro-1,1-dioxothienyl. Typically, monocyclic heterocycloalkyl groups have 3 to 7 members. Preferred 3 to 7 membered monocyclic heterocycloalkyl groups are those having 5 or 6 ring atoms. A heteroatom may be substituted with a protecting group known to those of ordinary skill in the art, for example, the hydrogen on a nitrogen may be substituted with a tert-butoxycarbonyl group. Furthermore, heterocycloalkyl groups may be optionally substituted with one or more substituents. In addition, the point of attachment of a heterocyclic ring to another group may be at either a carbon atom or a heteroatom of a heterocyclic ring. Only stable isomers of such substituted heterocyclic groups are contemplated in this definition.

As used herein the term “substituent” or “substituted” means that a hydrogen radical on a compound or group is replaced with any desired group that is substantially stable to reaction conditions in an unprotected form or when protected using a protecting group. Examples of preferred substituents are those found in the exemplary compounds and embodiments disclosed herein, as well as halogen (chloro, iodo, bromo, or fluoro); alkyl; alkenyl; alkynyl; hydroxy; alkoxy; nitro; thiol; thioether; imine; cyano; amido; phosphonato; phosphine; carboxyl; thiocarbonyl; sulfonyl; sulfonamide; ketone; aldehyde; ester; oxygen (—O); haloalkyl (e.g., trifluoromethyl); cycloalkyl, which may be monocyclic or fused or non-fused polycyclic (e.g., cyclopropyl, cyclobutyl, cyclopentyl, or cyclohexyl), or a heterocycloalkyl, which may be monocyclic or fused or non-fused polycyclic (e.g., pyrrolidinyl, piperidinyl, piperazinyl, morpholinyl, or thiazinyl), monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidinyl, benzimidazolyl, benzothiophenyl, or benzofuranyl); amino (primary, secondary, or tertiary); CO₂CH₃; CONH₂; OCH₂CONH₂; NH₂; SO₂NH₂; OCHF₂; CF₃; OCF₃; and such moieties may also be optionally substituted by a fused-ring structure or bridge, for example —OCH₂O—. These substituents may optionally be further substituted with a substituent selected from such groups. In certain embodiments, the term “substituent” or the adjective “substituted” refers to a substituent selected from the group consisting of an alkyl, an alkenyl, an alkynyl, an cycloalkyl, an cycloalkenyl, a heterocycloalkyl, an aryl, a heteroaryl, an aralkyl, a heteraralkyl, a haloalkyl, —C(O)NR₁₁R₁₂, —NR₁₃C(O)R₁₄, a halo, —OR₁₃, cyano, nitro, a haloalkoxy, —C(O)R₁₃, —NR₁₁R₁₂, —SR₁₃, —C(O)OR₁₃, —OC(O)R₁₃, —NR₁₃C(O)NR₁₁R₁₂, —OC(O)NR₁₁R₁₂, —NR₁₃C(O)OR₁₄, —S(O)rR₁₃, —NR₁₃S(O)rR₁₄, —OS(O)rR₁₄, S(O)rNR₁₁R₁₂, —O, —S, and —N—R₁₃, wherein r is 1 or 2; R₁₁ and R₁₂, for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₁₁ and R₁₂ taken together with the nitrogen to which they are attached is optionally substituted heterocycloalkyl or optionally substituted heteroaryl; and R₁₃ and R₁₄ for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl. In certain embodiments, the term “substituent” or the adjective “substituted” refers to a solubilising group.

The term “solubilising group” means any group which can be substantially ionized and that enables the compound to be soluble in a desired solvent, such as, for example, water or water-containing solvent. Furthermore, the solubilising group can be one that increases the compound or complex's lipophilicity. Typically, the solubilising group is selected from alkyl group substituted with one or more heteroatoms such as N, O, S, each optionally substituted with alkyl group substituted independently with alkoxy, amino, alkylamino, dialkylamino, carboxyl, cyano, or substituted with cycloheteroalkyl or heteroaryl, or a phosphate, or a sulfate, or a carboxylic acid. For example, by “solubilising group” it is referred herein to one of the following:
an alkyl, cycloalkyl, aryl, heretoaryl group comprising either at least one nitrogen or oxygen heteroatom or which group is substituted by at least one amino group or oxo group
an amino group which may be a saturated cyclic amino group which may be substituted by a group consisting of alkyl, alkoxycarbonyl, halogen, haloalkyl, hydroxyalkyl, amino, monoalkylamino, dialkylamino, carbamoyl, monoalkylcarbamoyl and dialkylcarbamoyl
one of the structures a) to i) shown below, wherein the wavy line and the arrow line correspond to the point of attachment to core structure of formula [A]

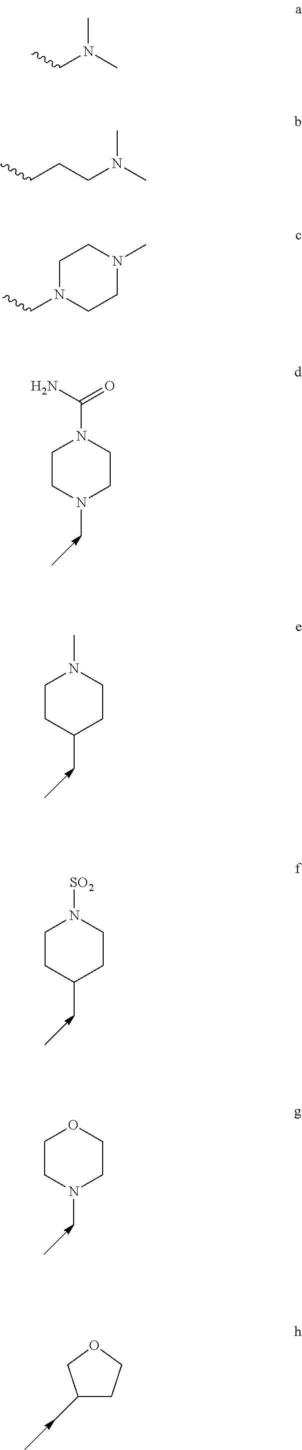

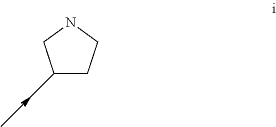

The term “cycloalkyl” means a saturated cyclic alkyl radical having from 3 to 10 carbon atoms. Representative cycloalkyls include cyclopropyl, 1-methylcyclopropyl, cyclobutyl, cyclopentyl, cyclohexyl, cycloheptyl, cyclooctyl, cyclononyl, and cyclodecyl. Cycloalkyl groups can be optionally substituted with one or more substituents.

The term “halogen” means —F, —Cl, —Br or —I.

### DETAILED DESCRIPTION

The present invention thus relates to a method for treating hepatic cancer in a subject in need thereof, wherein said method comprises administering a therapeutically effective amount of a tyrosine kinase inhibitor in combination with a therapeutically effective amount of at least one chemotherapeutic agent to the subject.

According to one embodiment, the present invention relates to a method for treating hepatic cancer in a subject in need thereof, wherein said method comprises administering to the subject a therapeutically effective amount of a tyrosine kinase inhibitor or a pharmaceutically acceptable salt or solvate thereof in combination with a therapeutically effective amount of a chemotherapeutic agent.

Indeed, the Applicant surprisingly demonstrated a synergetic effect of the combination of a tyrosine kinase inhibitor such as, for example, masitinib mesilate with a chemotherapeutic agent. First, the Applicant showed in in vitro tests that, despite the absence of effect of the compound alone, the administration of a tyrosine kinase inhibitor such as, for example, masitinib mesilate, sensitized cells to chemotherapeutic agents (see example 1). Second, the Applicant showed in in vivo data that the administration to a subject of a combination of a tyrosine kinase inhibitor such as, for example, masitinib mesilate, with a chemotherapeutic agent results in an increased overall survival (see example 2).

According to the invention, the method of the invention does not consist in administering a therapeutically effective amount of a tyrosine kinase inhibitor to the subject.

In one embodiment, the tyrosine kinase inhibitor of the invention is a c-Kit inhibitor. The present invention thus also relates to a c-Kit inhibitor for treating hepatic cancer.

In one embodiment, the method of the invention thus comprises administering a c-Kit inhibitor for treating hepatic cancer.

Examples of chemotherapeutic agents that may be used in combination with the tyrosine kinase inhibitor of the invention include, but are not limited to, sorafenib, doxorubicin, 5-fluorouracil, cisplatin, gemcitabine, doxorubicin, irinotecan, etoposide, vincristine, and mixtures thereof.

In one embodiment, the at least one chemotherapeutic agent is selected from the group comprising gemcitabine, doxorubicin, irinotecan, etoposide, vincristine, and mixtures thereof.

In one embodiment, the method of the invention comprises or consists in administering masitinib and gemcitabine to the subject. In another embodiment, the method of the invention comprises or consists in administering masitinib and doxorubicin to the subject. In another embodiment, the method of the invention comprises or consists in administering masitinib and irinotecan to the subject. In another embodiment, the method of the invention comprises or consists in administering masitinib and etoposide to the subject. In another embodiment, the method of the invention comprises or consists in administering masitinib and vincristine to the subject.

### Tyrosine Kinase Inhibitors

Tyrosine kinases are receptor type or non-receptor type proteins, which transfer the terminal phosphate of ATP to tyrosine residues of proteins thereby activating or inactivating signal transduction pathways. These proteins are known to be involved in many cellular mechanisms, which in case of disruption, lead to disorders such as abnormal cell proliferation and migration as well as inflammation. A tyrosine kinase inhibitor is a drug that inhibits tyrosine kinases, thereby interfering with signaling processes within cells. Blocking such processes can stop the cell growing and dividing.

In one embodiment, the tyrosine kinase inhibitor of the invention has the following formula [A]:

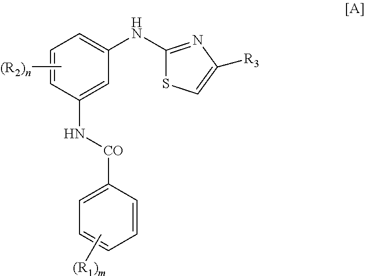

wherein
R₁ and R₂, are selected independently from hydrogen, halogen, a linear or branched alkyl, cycloalkyl group containing from 1 to 10 carbon atoms, trifluoromethyl, alkoxy, cyano, dialkylamino, and a solubilising group, m is 0-5 and n is 0-4;
the group R₃ is one of the following:
   i. an aryl group such as phenyl or a substituted variant thereof bearing any combination, at any one ring position, of one or more substituents such as halogen, alkyl groups containing from 1 to 10 carbon atoms, trifluoromethyl, cyano and alkoxy;
   ii. a heteroaryl group such as 2, 3, or 4-pyridyl group, which may additionally bear any combination of one or more substituents such as halogen, alkyl groups containing from 1 to 10 carbon atoms, trifluoromethyl and alkoxy;
   iii. a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy;
      or a pharmaceutically acceptable salt or solvate thereof

In one embodiment the tyrosine kinase inhibitor of the invention has the general formula [B],

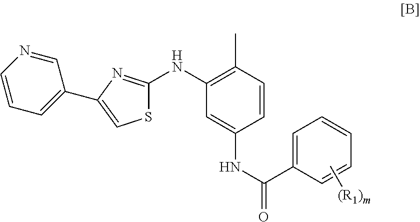

wherein:
R₁ is selected independently from hydrogen, halogen, a linear or branched alkyl, cycloalkyl group containing from 1 to 10 carbon atoms, trifluoromethyl, alkoxy, amino, alkylamino, dialkylamino, solubilising group.
m is 0-5,
   or a pharmaceutically acceptable salt or solvate thereof

In one embodiment, the tyrosine kinase inhibitor of formula [B] is masitinib or a pharmaceutically acceptable salt or solvate thereof, more preferably masitinib mesilate.

The present invention thus also relates to masitinib or a pharmaceutically acceptable salt or solvate thereof, more preferably masitinib mesilate for treating hepatic cancer.

According to one embodiment, the method of the invention thus comprises administering masitinib or a pharmaceutically acceptable salt or solvate thereof, more preferably masitinib mesilate, for treating hepatic cancer.

Pharmaceutically acceptable salts preferably are pharmaceutically acceptable acid addition salts, like for example inorganic acids, such as hydrochloric acid, sulfuric acid or a phosphoric acid, or suitable organic carboxylic or sulfonic acids, for example aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxy-benzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethane-sulfonic, in particular methanesulfonic acid, or aromatic sulfonic acids, for example benzene-, p-toluene- or naphthalene-2-sulfonic acid.

Unless otherwise indicated, references to “mesilate” are used in the present invention to refer to a salt of methanesulfonic acid with a named pharmaceutical substance (such as compounds of formula [A] or [B]). Use of mesilate rather than mesylate is in compliance with the INNM (International nonproprietary names modified) issued by WHO (e.g. World Health Organization (February 2006). International Nonproprietary Names Modified. INN Working Document 05.167/3. WHO.). For example, masitinib mesilate means the methanesulfonic acid salt of masitinib.

Preferably, “masitinib mesilate” means the orally bioavailable mesilate salt of masitinib—CAS 1048007-93-7 (MsOH); C28H30N6OS.CH3SO3H; MW 594.76:

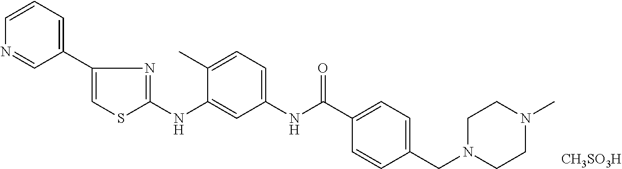

The chemical name for masitinib is 4-(4-methylpiperazin-1-ylmethyl)-N-[4-methyl-3-(4-pyridin-3ylthiazol-2-ylamino)phenyl]benzamide-CAS number 790299-79-5. Masitinib was described in U.S. Pat. No. 7,423,055 and EP1525200B1. A detailed procedure for the synthesis of masitinib mesilate is given in WO2008/098949.

Masitinib is a small molecule selectively inhibiting specific tyrosine kinases such as c-Kit, PDGFR, Lyn, Fyn and to a lesser extent the fibroblast growth factor receptor 3 (FGFR3), without inhibiting, at therapeutic doses, kinases associated with known toxicities (i.e. those tyrosine kinases or tyrosine kinase receptors attributed to possible tyrosine kinase inhibitor cardiac toxicity, including ABL, KDR and Src) (Dubreuil et al., 2009, PLoS ONE 2009.4(9):e7258).

The strong inhibitory effect of masitinib on wild-type and juxtamembrane-mutated c-Kit receptors results in cell cycle arrest and apoptosis of cell lines dependent on c-Kit signaling (Dubreuil et al., 2009, PLoS ONE, 4(9):e7258). In vitro, masitinib demonstrated greater activity and selectivity against c-Kit than imatinib, inhibiting recombinant human wild-type c-Kit with an half inhibitory concentration (IC₅₀) of 200±40 nM and blocking stem cell factor-induced proliferation and c-Kit tyrosine phosphorylation with an IC₅₀ of 150±80 nM in Ba/F3 cells expressing human or mouse wild-type c-Kit.

### Hepatic Cancer

In one embodiment, hepatic cancer is primary hepatic cancer, preferably hepatocellular carcinoma (HCC).

In one embodiment, the method of the invention is for treating unresectable HCC. Unresectable cancers include metastatic cancers and localized unresectable cancers, i.e. cancers that have not spread to the lymph nodes or distant organs but cannot be completely removed by surgery. There are several reasons why it might not be possible to safely remove a localized liver cancer. For example, if the non-cancerous part of the liver is not healthy (because of cirrhosis, for example), surgery might not leave enough liver tissue for it to function properly. Or curative surgery may not be possible if cancer is spread throughout the liver or is close to the area where the liver meets the main arteries, veins, and bile ducts.

In one embodiment, the method of the invention is for treating metastatic HCC. In another embodiment, the method of the invention is for treating localized unresectable HCC.

In one embodiment, hepatic cancer is advanced hepatic cancer. In one embodiment, the term “advanced hepatic cancer” corresponds to advanced hepatic cancer according to the BCLC (Barcelona Clinic Liver Cancer) staging.

The BCLC staging system uses variables related to tumor stage, liver functional status, physical status, and cancer-related symptoms. It comprises 5 stages, as shown in the Table below.

| | Performance | | |
|---|---|---|---|
| | status test | Tumour | |
| BCLC stage | score | characteristics | Liver function |
| 0 | 0 | Single tumour less | No increased |
| (very early) | | than 2 cm | pressure in the |
| | | | portal vein and |
| | | | normal bilirubin |
| | | | levels |
| A1 | 0 | Single tumour less | No increased |
| (early) | | than 5 cm | pressure in the |
| | | | portal vein |
| A2 | 0 | Single tumour less | Increased portal |
| (early) | | than 5 cm | vein pressure and |
| | | | normal bilirubin |
| | | | levels |
| A3 | 0 | Single tumour less | Increased portal |
| (early) | | than 5 cm | vein pressure |
| | | | and increased |
| | | | bilirubin levels |
| A4 | 0 | Three tumours, all | None applicable |
| | | less than 3 cm | |
| B | 0 | Large, multifocal | Child-Pugh A-B |
| (intermediate) | | tumour | |
| C | 1-2 | Tumour invades the | Child-Pugh A-B |
| (advanced) | | blood vessels or the | |
| | | cancer has spread to | |
| | | other sites | |
| D | 3-4 | Any tumour | Child-Pugh C |
| (End stage) | | | |

### Subject

In one embodiment, the subject was not treated previously with another treatment for hepatic cancer (i.e. the method of treatment of the invention is the first line treatment).

In another embodiment, the subject previously received one, two or more other treatment(s) for hepatic cancer (i.e. the method of treatment of the invention is a second line of treatment, a third line of treatment or more). In one embodiment, the subject previously received one or more other treatment(s) for hepatic cancer, but was unresponsive or did not respond adequately to these treatments, which means that there is no, or too low, therapeutic benefit induced by these treatments. Therapeutic benefits may include the fact of (1) slowing down or stopping the progression, aggravation, or deterioration of one or more symptoms of hepatic cancer; (2) bringing about ameliorations of the symptoms of hepatic cancer; (3) reducing the severity or incidence of hepatic cancer; or (4) curing hepatic cancer.

Examples of treatment for hepatic cancer include, but are not limited to, hepatectomy, liver transplant, tumor ablation (radiofrequency ablation (RFA), ethanol (alcohol) ablation, microwave thermotherapy or cryosurgery), embolization therapy (arterial embolization, chemoembolization, or radioembolization), radiation therapy, or treatment with sorafenib, doxorubicin, 5-fluorouracil, and cisplatin.

In one embodiment, the patient has not undergone liver transplantation. In another embodiment, the patient has previously undergone liver transplantation.

### Doses

In one embodiment, the therapeutically effective amount of a tyrosine kinase inhibitor or a pharmaceutically acceptable salt or solvate thereof ranges from about 1 to about 20 mg/kg/day, preferably from about 3 to about 12 mg/kg/day, and more preferably from about 4.5 to about 9 mg/kg/day. In one embodiment, the therapeutically effective amount of a tyrosine kinase inhibitor or a pharmaceutically acceptable salt or solvate thereof is of about 4.5 mg/kg/day or of about 6 mg/kg/day or of about 7.5 mg/kg/day or of about 9 mg/kg/day.

Unless otherwise indicated, any dose indicated herein refers to the amount of active ingredient as such, not to its salt form. Therefore, given that the tyrosine kinase inhibitor dose in mg/kg/day used in the described dose regimens refers to the amount of active ingredient tyrosine kinase inhibitor, compositional variations of a pharmaceutically acceptable salt of tyrosine kinase inhibitor will not change the said dose regimens.

In one embodiment, the tyrosine kinase inhibitor or a pharmaceutically acceptable salt or solvate thereof is orally administered.

In one embodiment, the tyrosine kinase inhibitor or a pharmaceutically acceptable salt or solvate thereof is administered once or twice a day.

In one embodiment, the therapeutically effective amount of a chemotherapeutic agent ranges from about 1 to 500 mg/m², preferably from about 25 to about 250 mg/m², and more preferably from about 45 to about 180 mg/m².

In one embodiment, the therapeutically effective amount of etoposide ranges from about 1 to 200 mg/m², preferably from about 25 to about 100 mg/m², and more preferably from about 45 to about 60 mg/m².

In one embodiment, the therapeutically effective amount of irinotecan ranges from about 50 to 500 mg/m², preferably from about 100 to about 250 mg/m², and more preferably from about 135 to about 180 mg/m².

In one embodiment, the chemotherapeutic agent is injected, such as, for example, by intravenous injection or infusion.

In one embodiment, the chemotherapeutic agent is administered once a day, or 1, 2, 3, 4, 5, 6, 7 times per week, or 1, 2, 3, 4, 5, 6, 7 times per two weeks or 1, 2, 3, 4, 5, 6, 7 times per months. In one embodiment, the administration schedule may include days or weeks periods wherein the chemotherapeutic agent is not administered. For example, the chemotherapeutic agent may be administered on the first day of each week, or on the first day of week 1 and week 2 of a 3 weeks cycle, or every days of a week followed by a 7 days rest period, and the like. The skilled artisan may easily adapt the administration schedule, according, for example, to the previous treatment history of the patient, to the severity of the disease to be treated, to the nature of the chemotherapeutic agent and the like.

### Medicament and Pharmaceutical Composition

Another object of the invention is a composition comprising a tyrosine kinase inhibitor or a pharmaceutically acceptable salt or solvate thereof and a chemotherapeutic agent. In one embodiment, the tyrosine kinase inhibitor is masitinib, preferably masitinib mesilate. In one embodiment, the chemotherapeutic agent is selected from gemcitabine, doxorubicin, irinotecan, etoposide, vincristine and mixtures thereof.

In one embodiment, the composition of the invention comprises or consists in masitinib and gemcitabine. In another embodiment, the composition of the invention comprises or consists in masitinib and doxorubicin. In another embodiment, the composition of the invention comprises or consists in masitinib and irinotecan. In another embodiment, the composition of the invention comprises or consists in masitinib and etoposide. In another embodiment, the composition of the invention comprises or consists in masitinib and vincristine.

Another object of the invention is a pharmaceutical composition comprising a tyrosine kinase inhibitor or a pharmaceutically acceptable salt or solvate thereof and a chemotherapeutic agent, in combination with at least one pharmaceutically acceptable carrier. In one embodiment, the tyrosine kinase inhibitor is masitinib, preferably masitinib mesilate. In one embodiment, the chemotherapeutic agent is selected from gemcitabine, doxorubicin, irinotecan, etoposide, vincristine and mixtures thereof.

In one embodiment, the pharmaceutical composition of the invention comprises or consists in masitinib and gemcitabine in combination with at least one pharmaceutically acceptable carrier. In another embodiment, the pharmaceutical composition of the invention comprises or consists in masitinib and doxorubicin in combination with at least one pharmaceutically acceptable carrier. In another embodiment, the pharmaceutical composition of the invention comprises or consists in masitinib and irinotecan in combination with at least one pharmaceutically acceptable carrier. In another embodiment, the pharmaceutical composition of the invention comprises or consists in masitinib and etoposide in combination with at least one pharmaceutically acceptable carrier. In another embodiment, the pharmaceutical composition of the invention comprises or consists in masitinib and vincristine in combination with at least one pharmaceutically acceptable carrier.

Another object of the invention is a medicament comprising a tyrosine kinase inhibitor or a pharmaceutically acceptable salt or solvate thereof and a chemotherapeutic agent.

In one embodiment, the tyrosine kinase inhibitor is masitinib, preferably masitinib mesilate. In one embodiment, the chemotherapeutic agent is selected from gemcitabine, doxorubicin, irinotecan, etoposide, vincristine and mixtures thereof.

In one embodiment, the medicament of the invention comprises or consists in masitinib and gemcitabine. In another embodiment, the medicament of the invention comprises or consists in masitinib and doxorubicin. In another embodiment, the medicament of the invention comprises or consists in masitinib and irinotecan. In another embodiment, the medicament of the invention comprises or consists in masitinib and etoposide. In another embodiment, the medicament of the invention comprises or consists in masitinib and vincristine.

Another object of the invention is a kit of part comprising two parts, wherein the first part comprises a tyrosine kinase inhibitor or a pharmaceutically acceptable salt or solvate thereof and wherein the second part comprises a chemotherapeutic agent. In one embodiment, the tyrosine kinase inhibitor is masitinib, preferably masitinib mesilate. In one embodiment, the chemotherapeutic agent is selected from gemcitabine, doxorubicin, irinotecan, etoposide, vincristine and mixtures thereof.

In one embodiment, the first part of the kit of part of the invention comprises masitinib or a pharmaceutically acceptable salt or solvate thereof, preferably masitinib mesilate, and the second part of the kit of part of the invention comprises a chemotherapeutic agent selected from gemcitabine, doxorubicin, irinotecan, etoposide, vincristine and mixtures thereof.

In one embodiment, the first part of the kit of part of the invention comprises masitinib and the second part comprises gemcitabine. In another embodiment, the first part of the kit of part of the invention comprises masitinib and the second part comprises doxorubicin. In another embodiment, the first part of the kit of part of the invention comprises masitinib and the second part comprises irinotecan. In another embodiment, the first part of the kit of part of the invention comprises masitinib and the second part comprises etoposide. In another embodiment, the first part of the kit of part of the invention comprises masitinib and the second part comprises vincristine.

In one embodiment of the invention, the composition, pharmaceutical composition, medicament or kit of part of the invention comprises an amount of a tyrosine kinase inhibitor ranging from about 10 to about 500 mg, preferably from about 50 to about 300 mg, and more preferably from about 100 to about 200 mg.

In one embodiment of the invention, the composition, pharmaceutical composition, medicament or kit of part of the invention comprises an amount of masitinib ranging from about 10 to about 500 mg, preferably from about 50 to about 300 mg, and more preferably from about 100 to about 200 mg. In one embodiment, the composition, pharmaceutical composition, medicament or kit of part of the invention comprises an amount of masitinib of about 100 mg (corresponding to an amount of masitinib mesilate of about 119.3 mg). In another embodiment, the composition, pharmaceutical composition, medicament or kit of part of the invention comprises an amount of masitinib of about 200 mg (corresponding to an amount of masitinib mesilate of about 238.5 mg).

In one embodiment, the composition, pharmaceutical composition, medicament of the invention or the first and/or second part of the kit of part of the invention is in a form adapted for oral administration.

Examples of forms adapted for oral administration include, but are not limited to, tablets, orodispersing tablets, effervescent tablets, powders, granules, pills (including sugarcoated pills), dragees, capsules (including soft gelatin capsules), syrups, liquids, gels or other drinkable solutions, suspensions, slurries, liposomal forms and the like.

In one embodiment, the composition, pharmaceutical composition, medicament of the invention or the first and/or second part of the kit of part of the invention is in a form adapted for injection, such as, for example, for intramuscular, subcutaneous, intradermal, transdermal or intravenous injection or infusion.

Examples of forms adapted for injection include, but are not limited to, solutions, such as, for example, sterile aqueous solutions, dispersions, emulsions, suspensions, solid forms suitable for using to prepare solutions or suspensions upon the addition of a liquid prior to use, such as, for example, powder, liposomal forms and the like.

In one embodiment, the part of the kit of part comprising the tyrosine kinase inhibitor or a pharmaceutically acceptable salt or solvate thereof is in a form adapted for oral administration, while the second part of the kit of part (comprising the chemotherapeutic agent) is in a form adapted for injection.

The present invention further relates to a composition, a pharmaceutical composition, a medicament or a kit of part as described hereinabove for treating hepatic cancer, or for use in treating hepatic cancer.

In one embodiment of the invention, the composition, pharmaceutical composition, medicament or kit of part as described hereinabove is for use in the method for treating hepatic cancer of the invention.

According to one embodiment, the method of the invention comprises administering the composition, pharmaceutical composition, medicament or kit of part as described hereinabove for treating hepatic cancer.

### Mechanism

Masitinib is a small molecule drug, selectively inhibiting specific tyrosine kinases such as c-Kit, platelet-derived growth factor receptor (PDGFR), LYN, and FYN, without inhibiting, at therapeutic doses, kinases associated with known toxicities (i.e. those tyrosine kinases or tyrosine kinase receptors attributed to possible tyrosine kinase inhibitor cardiac toxicity, including ABL, KDR and Src) [Dubreuil, 2009].

In one embodiment, the method of the invention comprises inhibiting tyrosine kinases, preferably selected from the group consisting of c-Kit, LYN, FYN and PDGFR α and β, thereby treating hepatic cancer.

The present invention thus also relates to a method for inhibiting tyrosine kinases, preferably selected from the group consisting of c-Kit, LYN, FYN and PDGFR α and β in a hepatic cancer patient, thereby treating hepatic cancer, wherein said method comprises administering a therapeutically effective amount of masitinib or a pharmaceutically acceptable salt or solvate thereof.

In one embodiment, the method of the invention comprises inhibiting c-Kit. In one embodiment, the method of the invention comprises inhibiting LYN. In one embodiment, the method of the invention comprises inhibiting FYN. In one embodiment, the method of the invention comprises inhibiting PDGFR α and β, in particular inhibiting the in vitro protein kinase activity of PDGFR-α and β.

The main kinase target of masitinib is c-Kit, for which it has been shown to exert a strong inhibitory effect on wild-type and juxtamembrane-mutated c-Kit receptors, resulting in cell cycle arrest and apoptosis of cell lines dependent on c-Kit signaling [Dubreuil et al., 2009, PLoS ONE, 4(9):e7258]. In vitro, masitinib demonstrated high activity and selectivity against c-Kit, inhibiting recombinant human wild-type c-Kit with an half inhibitory concentration (IC50) of 200±40 nM and blocking stem cell factor-induced proliferation and c-Kit tyrosine phosphorylation with an IC50 of 150±80 nM in Ba/F3 cells expressing human or mouse wild-type c-Kit. In addition to its anti-proliferative properties, masitinib can also regulate the activation of mast cells through its targeting of Lyn and Fyn, key components of the transduction pathway leading to IgE induced degranulation [Gilfillan et al., 2006, Nat Rev Immunol, 6:218-230] [Gilfillan et al., 2009, Immunological Reviews, 228:149-169]. This can be observed in the inhibition of FcεRI-mediated degranulation of human cord blood mast cells [Dubreuil et al., 2009, PLoS ONE; 4(9):e7258]. Masitinib is also an inhibitor of PDGFR α and β receptors. Recombinant assays show that masitinib inhibits the in vitro protein kinase activity of PDGFR-α and β with IC50 values of 540±60 nM and 800±120 nM. In Ba/F3 cells expressing PDGFR-α, masitinib inhibited PDGF-BB-stimulated proliferation and PDGFR-α tyrosine phosphorylation with an IC50 of 300±5 nM.

In oncology indications for which the tyrosine kinase targets of masitinib are not the main oncogenic drivers, the main mode of action of masitinib is through modulation of the immune response. Experimental data indicate that masitinib is capable of modulating the immune response in such a way as to positively impact on physiological disturbances such as oxidative stress [Adenis A, et al. Ann Oncol. 2014 September; 25(9):1762-9]. In particular, masitinib induces an anti-tumoral Th1 immune response via recruitment of macrophages with a potential antitumoral activity within the tumor and also modulates the tumor microenvironment through its inhibition of mast cell activity with reduced release of M2-polarizing cytokines (protumoral), as well as other factors favoring metastasis and angiogenesis. Subsequent antitumoral activity within the tumor and tumor microenvironment confers conditions conducive to retarding aggressiveness and dissemination of the tumor in a manner independent of association with any particular active chemotherapeutic agent.

More specifically, recent experimental data demonstrate that masitinib induces an anti-tumoral Th1 immune response, due to the following mechanisms of action: (i) masitinib acts on macrophage, by increasing both the release of chemoattractants which attracts macrophages to the tumor site (such as, for example, CCL2), and the expression of M1-polarizing cytokines, such as, for example, CXCL9 and CXCL10; (ii) masitinib inhibits mast cell proliferation and degranulation and thereby reduces the release of M2-polarizing cytokines, as well as other factors favoring metastasis and angiogenesis (such as VEGF); and (iii) masitinib increases cytotoxic NK activity and IFN gamma release through its interaction with dendritic cells.

In one embodiment, the method of the invention comprises inducing an anti-tumoral Th1 immune response, thereby treating hepatic cancer.

The present invention thus also relates to a method for inducing an anti-tumoral Th1 immune response in a hepatic cancer patient, thereby treating hepatic cancer, wherein said method comprises administering a therapeutically effective amount of masitinib or a pharmaceutically acceptable salt or solvate thereof.

In one embodiment, the method of the invention comprises increasing the release of chemoattractants which attracts macrophages to the tumor site (such as, for example, CCL2), and/or increasing the expression of M1-polarizing cytokines, such as, for example, CXCL9 and CXCL10.

In one embodiment, the method of the invention comprises inhibiting mast cell proliferation and degranulation and thereby reducing the release of M2-polarizing cytokines, as well as other factors favoring metastasis and angiogenesis (such as VEGF).

In one embodiment, the method of the invention comprises increasing cytotoxic NK activity and IFN gamma release.

In one embodiment, the method of the invention comprises (i) inhibiting tyrosine kinases, preferably selected from the group consisting of c-Kit, LYN, FYN and PDGFR α and β and (ii) inducing an anti-tumoral Th1 immune response, thereby treating hepatic cancer.

The present invention thus also relates to a method for (i) inhibiting tyrosine kinases, preferably selected from the group consisting of c-Kit, LYN, FYN and PDGFR α and β and (ii) inducing an anti-tumoral Th1 immune response, in a hepatic cancer patient, thereby treating hepatic cancer, wherein said method comprises administering a therapeutically effective amount of masitinib or a pharmaceutically acceptable salt or solvate thereof.

According to one embodiment, the present invention relates to a method for inhibiting tyrosine kinases selected from the group consisting of c-Kit, LYN, FYN and PDGFR α and β and for inducing an anti-tumoral Th1 immune response, in a hepatic cancer patient, thereby treating hepatic cancer, wherein said method comprises administering a therapeutically effective amount of masitinib or a pharmaceutically acceptable salt or solvate thereof in combination with a therapeutically effective amount of a chemotherapeutic agent.

### EXAMPLES

The present invention is further illustrated by the following examples.

### Example 1

### Masitinib Sensitized Hepatoma Cell Lines to Chemotherapies

### Materials and Methods

### Compounds

Masitinib (having the molecular formula C₂₈H₃₀N₆OS.CH₄O₃S) presents as a white powder. Stock solution of 20 mM in DMSO was stored at −80° C. Gemcitabine (2′,2′,-dofluoro-2′,-deoxycytidine) was from Eli Lilly and is a nucleoside analogue of deoxycytidine that interferes with DNA synthesis. The other agents were purchased from Sigma Aldrich Corporation and are a poison of microtubules (Vincristin), an anti-topoisomerase I (Irinotecan), an anti-topoisomerase II (Etoposide) and doxorubicin (anthracycline antibiotic). These agents are commonly used as treatment for various tumor types either as single agent or in combination regimens.

### Cell Culture

Hepatoma cell lines PLC-PRF5 and HepG2 (purchased from Cell Line Service, Germany) were cultured as monolayers in DMEM Glutamax and DMEM:F12 (1/1 mixture) Glutamax respectively, supplemented with 100U/mL penicillin and 100 μg/mL streptomycin, and 10% v/v heat-inactivated foetal calf serum (Eurobio ref CVFSVF00-01 Lot S35531-1135) under standard culture conditions (5% CO2, 95% air in humidified chamber at 37° C.). During proliferation assays, all cells were grown in medium containing 1% FCS.

### Experimental Design

Colorimetric cell proliferation and viability assay (reagent CellTiter-Blue purchased from Promega cat N°G8081)—Cells were washed once, resuspended in DMEM/DMEM:F12 1% FCS and then plated at 1.10⁴/50 μl per well of a 96 well plate. Drug dilutions were prepared in a 96 well plate and obtained by sequential dilutions of masitinib or gemcitabine in DMEM/DMEM:F12 1% FCS. Treatment was started by the addition of 50 μl of a 2× concentrated drug solution to a final volume of 100 μl. For treatment with combinations of masitinib and cytotoxic agents, the cells were first resuspended in medium DMEM/DMEM:F12 1% FCS containing masitinib at the concentrations of 0, 2, 5 and 10 μM, plated as before in 96 wells plates and placed in the incubator overnight (o/n) before treatment with cytotoxic agents. Cytotoxic agent treatment was initiated by addition of 50 μl of a 2× drug dilution (and containing the respective masitinib drug concentration) to a final volume of 100 μl. Masitinib final concentrations remained 0, 2, 5 and 10 μM. After incubating for 72 hours at 37° C., 10 μl of a ½ dilution of CellTiter-Blue reagent was added to each well and the plates were returned to the incubator for an additional 4 hours. The fluorescence intensity from the CellTiter-Blue reagent is proportional to the number of viable cells and data were recorded (544Ex/590Em) using a POLARstar OMEGA microplate reader (BMG Labteck Sarl). A background control without cells was used as a blank. The positive control of the assay corresponds to the cell proliferation obtained in the absence of drug treatment (100% proliferation). Each sample was done in duplicate, the absorbance values were transferred to an excel file, the average and standard deviation of the duplicates were calculated and expressed as a percentage of the proliferation obtained in absence of treatment. The results presented are representative of a minimum of 3 experiments. The sensitization factor/Index is calculated by dividing the IC₅₀ of the chemotherapeutic agent alone by the IC₅₀ of the chemotherapeutic agent used in combination with masitinib mesilate.

### Results

In order to assess the benefits of using masitinib in combination therapy for cancer treatment, preclinical studies involving tumour cell lines were performed. The project consisted to evaluate the ability of masitinib to sensitize hepatoma cell lines PLC-PRF5 and HepG2 to cytotoxic agents using in vitro proliferation assays.

We used a large panel of cytotoxic agents that exert their cytotoxicity through different mechanisms. These agents included the conventionnal chemotherapies Doxorubicin (DOX), Gemcitabine (GCB) as well as non-standard chemotherapeutic agents such as Irinotecan (CPT-11), Etoposide (VP-16), and Vincristin (VINC).

### Masitinib Mesilate is not Active as Single Agent

Hepatoma cell lines PLC-PRF5 and HepG2 were first analyzed for their sensitivity to masitinib mesilate when used as single agent. This analysis showed that hepatoma cell lines were not sensitive to masitinib mesilate (IC₅₀>5 μM) suggesting that proliferation/survival of the cell line examined may not be dependent on the expression of masitinib main targets PDGFRβ and c-Kit. Based on these data, masitinib mesilate was used at concentrations of 5 and 10 μM in the following combinatory experiments.

### Masitinib Mesilate Sensitizes Hepatoma Cell Lines to Gemcitabine

To determine the IC₅₀ of gemcitabine as single agent or in association with masitinib mesilate, hepatoma cell lines grown in 1% FCS were pre-treated with solvent control (DMSO) or masitinib for about 12-16 hours before being exposed to different doses of the chemotherapeutic agent.

Results are shown in Table 1.

TABLE 1
Masitinib mesilate sensitizes hepatoma cell line
to Gemcitabine (GCB)

| IC₅₀ μM | GCB | GCB + | |
|---|---|---|---|
| Cell lines | μM | Masitinib mesilate | Sensitization factor |
| HepG2 | >100 | 10-100 | 1-10 |

SI = Sensitization Index

The HepG2cell line is thus sensitized to the action of the chemotherapeutic agent gemcitabine by the addition of masitinib mesilate.

### Masitinib Mesilate Sensitizes Hepatoma Cell Lines to Doxorubicin

We next assessed the ability of masitinib mesilate to sensitize hepatoma cell line to the action of the anthracycline doxorubicin (Adriamycin). Summary of the results is presented in Table 2.

TABLE 2
Masitinib mesilate sensitizes hepatoma cell line
to Doxorubicin (DOX)

| IC₅₀ (μM) | DOX | DOX + | |
|---|---|---|---|
| Cell lines | μM | Masitinib mesilate | Sensitization factor |
| HepG2 | 5 | 1 | 5 |

SI = Sensitization Index

The addition of masitinib mesilate does enhance the response of HepG2 cell line to the cytotoxic agent.

### Masitinib Mesilate Sensitizes Hepatoma Cell Lines to Vincristin

We next examined the ability of masitinib mesilate to sensitize hepatoma cell lines to the action of the alkaloid agent vincristin (VINC). Results are shown in Table 3.

TABLE 3
Masitinib mesilate sensitizes hepatoma cell line
to Vincristin (VINC)

| IC50 μM | VINC | VINC + | |
|---|---|---|---|
| Cell lines | μM | Masitinib mesilate | Sensitization factor |
| HepG2 | 0.1-1 | 0.1 | 1-10 |
| PLC-PRF5 | 0.1-1 | 0.01 | >10 |

SI = Sensitization Index

Although the cell lines exhibit partial resistance to vincristine, the addition of masitinib mesilate significantly potentiates the action of the chemotherapeutic agent.

### Masitinib Mesilate Sensitizes Hepatoma Cell Lines to Etoposide

We next tested the ability of masitinib mesilate to sensitize hepatoma cell lines to the action of anti-topoisomerase II agent etoposide (VP-16). Summary of the results is presented in Table 4.

TABLE 4
Masitinib mesilate sensitizes hepatoma cell lines
to Etoposide (VP-16)

| IC50 μM | | VP-16 + | |
|---|---|---|---|
| Cell lines | VP-16 | masitinib mesilate | SI |
| HepG2 | >100 | 30 | >3 |
| PLC-PRF5 | 50 | 1-10 | 5-50 |

SI = Sensitization Index

Interestingly both cell lines were sensitized to etoposide when masitinib mesilate was added. The presence of masitinib lowers the IC₅₀ of etoposide to clinically achievable concentrations (Approximate Cmax measured in plasma of 34 μM).

### Masitinib Mesilate Sensitizes Hepatoma Cell Lines to Irinotecan

We next tested the ability of masitinib mesilate to sensitize hepatoma cell lines to the action of anti-topoisomerase I agent irinotecan (CPT-11). Summary of the results is presented in Table 5.

TABLE 5
Masitinib mesilate sensitizes hepatoma cell lines
to Irinotecan (CPT-11)

| IC50 μM | | CPT-11 + | |
|---|---|---|---|
| Cell lines | CPT-11 | masitinib mesilate | SI |
| HepG2 | 100 | 10-20 | 5-10 |
| PLC-PRF5 | 100 | 10 | 10 |

SI = Sensitization Index

Interestingly both cell lines appear to be resistant to irinotecan and a good sensitization is observed when masitinib mesilate was added. The presence of masitinib mesilate lowers the IC₅₀ of irinotecan to clinically achievable concentrations (Approximate Cmax measured in plasma of 1-10 μM).

These results thus demonstrate that, surprisingly, masitinib mesilate is able to sensitize hepatoma cell line to cytotoxic agents in vitro, despite its absence of activity when used alone. Therefore, these results highlight the synergistic effect of the combination of masitinib mesilate and cytotoxic agents.

### Example 2

### Treatment of HCC with a Combination of Masitinib Mesilate and Irinotecan

A prospective, multicenter, open-label, randomized, uncontrolled, phase ½ clinical study has been conducted to evaluate efficacy and safety of masitinib mesilate in association with irinotecan in patients suffering from advanced hepatocellular carcinoma (according to the BCLC staging) and who relapsed after a first-line therapy with sorafenib.

### Methodology

Six patients resistant to a first line of chemotherapy with the single agent sorafenib have been enrolled. In this open-label study, masitinib mesilate was administered orally at the daily dose of 6 mg/kg or 7.5 mg/kg in two intakes, in combination with irinotecan infused at the dose of 180 mg/m² once every two weeks.

### Preliminary Results

Overall survival (OS) is defined as the time from first treatment intake to the date of documented death. If death was not observed, data on OS were censored at the last date patient was known to be alive. OS was analyzed using Kaplan-Meier and was given with its confidence interval (CI) of 95%.

In this study, last available analysis shows overall survival with masitinib mesilate in combination with irinotecan is 9.0 months while the benchmark for a second-line of chemotherapy (L2) is 5 months. Summary of the results is presented in Table 6.

TABLE 6
Overall Survival in the ITT population

| | Median OS (months) | [95% CI] |
|---|---|---|
| Benchmark L2* | 5 | |
| Masitinib + Irinotecan | 9.0 | [6.2-NR] |

*Study

These preliminary results demonstrate that the administration of a combination of masitinib mesilate and irinotecan to patients suffering from advanced hepatocellular carcinoma increases overall survival.

### Example 3

### The Data and Safety Monitoring Board Recommends the Continuation of the Phase 2 Study with Masitinib in Advanced Hepatocellular Carcinoma Based on Safety and Efficacy Data

AB Science SA (NYSE Euronext—FR0010557264—AB), a pharmaceutical company specialized in research, development and marketing of protein kinase inhibitors (PKIs), announces that the external Data and Safety Monitoring Board (DSMB) has recommended the continuation of its phase 2 study of masitinib in advanced hepatocellular carcinoma based upon review of the latest safety and efficacy data. The DSMB was created as part of the Company's clinical study evaluating masitinib in the treatment of advanced hepatocellular carcinoma.

The objective of this phase 2 study is to evaluate the safety and efficacy of masitinib in combination with etoposide, or masitinib in combination with irinotecan in patients with advanced hepatocellular carcinoma and who relapsed after a first line therapy with sorafenib. The study primary endpoint is overall survival.

There are three objectives: to determine if at least one combination has a trend of superiority on overall survival as compared to the latest benchmark in this indication, to determine which combination has the best benefit/risk if any, to determine the best dose of both masitinib and chemotherapies. Those three objectives are considered pre requisite to move into phase 3.

This recommendation from the DSMB is encouraging because it confirms that the benefit risk balance for masitinib is positive based on the data currently generated in this study.

Dr. Yann TOUCHEFEU (Service d'Hépato-gastroentérologie, CHU Hotel-Dieu, Nantes-France), principal investigator of the study indicated that “There is a high unmet medical need for patients in second line treatment of advanced hepatocellular carcinoma. The median overall survival is around 5 months with current therapies. For that reason, if masitinib confirmed an acceptable safety profile and showed a trend of increased overall survival as compared with this current benchmark, phase 3 would be warranted and masitinib could provide an option for second-line treatment of patients with advanced hepatocellular carcinoma”.

There is a growing incidence of Hepatocellular carcinoma worldwide. The incidence was of 86,000 cases in the USA and Europe in 2008, and the mortality rate was of 78,640 cases. It is estimated that by 2020 the number of cases will reach 105,000 in these geographies.

Around 40% of patients have advanced Hepatocellular carcinoma (BCLC stage C). These patients bear a dismal prognosis and are eligible as first line treatment to Nevaxar (sorafenib) a multi kinase inhibitor. With, sorafenib, the median treatment time is around 5.5 months, and the median OS is 9.5 months in BCLC C patients.

There is currently no approved standard in second line of treatment after failure with sorafenib. Masitinib is therefore addressing a clear unmet medical need.

Around 60% of patients progressing after sorafenib usually can still take a second line of treatment. With this hypothesis the number of eligible patients for second line treatment of advanced hepatocellular carcinoma is estimated to be 25,000 per annum in Europe and USA by 2020.

### About Hepatocellular Carcinoma

Liver cancer is the fifth most common cancer (749,000 new cases), the third cause of cancer related death (692,000 cases). Hepatocellular carcinoma represents more than 90% of primary liver cancers.

Resection may benefit certain patients, albeit mostly transiently. Many patients are not candidates given the advanced stage of their cancer at diagnosis. In these patients, local ablative therapies, including radiofrequency ablation, chemoembolization, and potentially novel chemotherapeutic agents, may extend life and provide palliation. Only a fraction of all patients have access to transplantation

1. A method for treating hepatic cancer in a subject in need thereof, comprising administering to the subject a therapeutically effective amount of a tyrosine kinase inhibitor or a pharmaceutically acceptable salt or solvate thereof in combination with a therapeutically effective amount of a chemotherapeutic agent.

2. The method according to claim 1, wherein the tyrosine kinase inhibitor is an inhibitor of at least one kinase selected from the group consisting of c-Kit, Lyn, Fyn and PDGFR α and β.

3. The method according to claim 1, wherein the tyrosine kinase inhibitor is masitinib or a pharmaceutically acceptable salt or solvate thereof.

4. The method according to claim 1, wherein the tyrosine kinase inhibitor is masitinib mesilate.

5. The method according to claim 1, wherein the chemotherapeutic agent is selected from the group consisting of gemcitabine, doxorubicin, irinotecan, etoposide, vincristine and mixtures thereof.

6. The method according to claim 1, wherein hepatic cancer is primary hepatic cancer.

7. The method according to claim 1, wherein hepatic cancer is hepatocellular carcinoma (HCC).

8. The method according to claim 1, wherein hepatic cancer is unresectable and/or metastatic hepatocellular carcinoma (HCC).

9. The method according to claim 1, wherein hepatic cancer is advanced hepatic cancer according to the BCLC staging.

10. The method according to claim 1, wherein the therapeutically effective amount of the tyrosine kinase inhibitor or a pharmaceutically acceptable salt or solvate thereof ranges from about 4.5 mg/kg/day to about 9 mg/kg/day.

11. The method according to claim 1, wherein the tyrosine kinase or a pharmaceutically acceptable salt or solvate thereof inhibitor is orally administered.

12. The method according to claim 1, wherein the tyrosine kinase inhibitor or a pharmaceutically acceptable salt or solvate thereof is administered twice daily.

13. A method for inhibiting tyrosine kinases, selected from the group consisting of c-Kit, LYN, FYN and PDGFR α and β, and for inducing an anti-tumoral Th1 immune response, in a hepatic cancer patient, thereby treating hepatic cancer, wherein said method comprises administering a therapeutically effective amount of masitinib or a pharmaceutically acceptable salt or solvate thereof in combination with a therapeutically effective amount of a chemotherapeutic agent.

14. A composition comprising a tyrosine kinase inhibitor or a pharmaceutically acceptable salt or solvate thereof, and a chemotherapeutic agent.

15. The composition according to claim 14, wherein said tyrosine kinase inhibitor is masitinib mesilate, and said chemotherapeutic agent is selected from the group consisting of gemcitabine, doxorubicin, irinotecan, etoposide, vincristine and mixtures thereof.
